(19) United States
(12) Patent Application Publication
(10) Pub. No.: US 2005/0148802 A1
(43) Pub. Date: Jul. 7, 2005

(54) Method for producing polyetherols

(75) Inventors: Alexander Wartini, Heidelberg (DE); Matthias Dernbach, Dossenheim (DE); Claus Hackmann, Kirchheim (DE); Tilman Sirch, Schifferstadt (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: Basf Aktiengesellschaft

(21) Appl. No.: 10/513,323

(22) PCT Filed: May 21, 2003
(86) PCT No.: PCT/EP03/05306

(30) Foreign Application Priority Data
May 24, 2002 (DE) 10223054.4

(51) Int. Cl. C07C 43/32; C07C 43/30

(52) U.S. Cl. 568/591; 568/594

(57) ABSTRACT

The invention provides a process for preparing polyetherols of polyhydric alcohols by reacting an alkylene oxide with the appropriate polyhydric alcohol in the presence of a base and in the presence or absence of a solvent, wherein the polyhydric alcohol used has a formaldehyde acetal content of less than 500 ppm.

The present invention describes a process for preparing polyetherols of polyhydric alcohols by polyetherification of an alkylene oxide with the appropriate polyhydric alcohol in the presence of at least one preferably basic catalyst, with or without a solvent.

Polyhydric alcohols are those compounds which have more than one hydroxyl group, for example from 2 to 6, preferably from 2 to 4, more preferably 2 or 3 and in particular 3.

Such polyetherols are used, inter alia, as a starting product for producing polyurethanes, as a lubricant and as an intermediate for acrylates in numerous applications.

These applications require in particular colorless products having no inherent odor, low acid number and high storage stability.

The preparation of polyetherols by base-catalyzed reaction of an alkylene oxide with the appropriate alcohols in the presence of water or another solvent is well known.

The catalysts used are in general amines or else alkali metal hydroxides or alkoxides or hydrotalcite, preferably alkali metal hydroxides in water or mixtures thereof. Recently, double metal cyanide catalysts, frequently also referred to as DMC catalysts, have become more important in preparing polyether alcohols.

Since the polyetherols of polyhydric alcohols generally cannot be distillatively purified owing to their high boiling points, by-products remain in the end product and influence the further processing and/or quality both of the target ether and also of the subsequent products.

It is an object of the present invention to provide an economical process which facilitates the preparation of polyetherols of polyhydric alcohols on an industrial scale in high purity and in high yield in a simple manner and without additional assistants.

We have found that this object is achieved by a process for preparing polyetherols of polyhydric alcohols by reacting alkylene oxides with the appropriate polyhydric alcohol in the presence of at least one basic catalyst and in the presence or absence of a solvent, wherein the polyhydric alcohol used has a formaldehyde acetal content of less than 500 ppm.

The novel process has the following decisive advantage: the end product is substantially colorless. Color number variations between different production campaigns do not occur.

The polyhydric alcohols used may be, for example, trimethylolbutane, trimethylolpropane, trimethylolethane, neopentyl glycol, pentaerythritol, 2-ethyl-1,3-propanediol, 2-methyl-1,3-propanediol, glycerol, ditrimethylolpropane, dipentaerythritol, bisphenol A, bisphenol F, bisphenol B, bisphenol S, 2,2-bis(4-hydroxycyclohexyl)propane, 1,1-, 1,2-, 1,3- and 1,4-cyclohexanedimethanol, 1,2-, 1,3- or 1,4-cyclohexanediol, sorbitol, mannitol, diglycerol, threitol, erythritol, adonitol (ribitol), arabitol (lyxitol), xylitol or dulcitol (galactitol).

Preference is given to using those polyhydric alcohols in the process according to the invention which are obtained by reacting an aldehyde with formaldehyde and subsequently converting the aldehyde group to a hydroxyl group.

These include, for example, polyhydric alcohols of the formula (I): 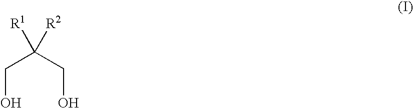
where
R¹, R² are each independently hydrogen, C₁-C₁₀-alkyl, C₁-C₁₀-hydroxyalkyl, carboxyl or C₁-C₄-alkoxycarbonyl, preferably hydrogen, hydroxymethyl or C₁-C₁₀-alkyl and more preferably hydroxymethyl or C₁-C₁₀-alkyl.

The alkyl radicals may each be straight-chain or branched.

Examples of R¹ and R² include hydrogen, methyl, ethyl, iso-propyl, n-propyl, n-butyl, isobutyl, sec-butyl, tert-butyl, n-pentyl, n-hexyl, n-heptyl, n-octyl, n-decyl, hydroxymethyl, carboxyl, methoxycarbonyl, ethoxycarbonyl or n-butoxycarbonyl, and preference is given to hydrogen, hydroxymethyl, methyl and ethyl, particular preference to hydroxymethyl, methyl and ethyl.

Examples of polyhydric alcohols of the formula (I) include trimethylolbutane, trimethylolpropane, trimethylolethane, neopentyl glycol, pentaerythritol, 2-ethyl-1,3-propanediol, 2-methyl-1,3-propanediol, 1,3-propanediol, dimethylolpropionic acid, methyl dimethylolpropionate, ethyl dimethylolpropionate, dimethylolbutyric acid, methyl dimethylolbutyrate or ethyl dimethylolbutyrate, and preference is given to neopentyl glycol, trimethylolpropane, pentaerythritol and dimethylolpropionic acid, particular preference to neopentyl glycol, trimethylolpropane and pentaerythritol, very particular preference to trimethylolpropane and pentaerythritol and in particular to trimethylolpropane.

Such polyhydric alcohols of the formula (I) are obtainable, for example, by reacting an aldehyde of the formula (II) 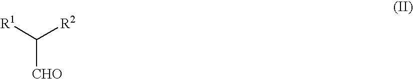
where R¹ and R² are each as defined above with formaldehyde and subsequently converting the aldehyde group to a hydroxyl group.

The polyhydric alcohols of the general formula (I) are obtained on an industrial scale by condensation of formaldehyde with higher, CH-acidic aldehydes (II) or with water and acrolein or 2-alkylacroleins. In this reaction, a distinction is drawn between two principal variants of carrying out the conversion of the aldehyde group to a hydroxyl group which are illustrated hereinbelow by the preparation of trimethylolpropane but are in no way limited thereto.

Firstly, there is the Cannizzaro process which is in turn divided into the inorganic and the organic Cannizzaro processes. In the inorganic variant, an excess of formaldehyde is reacted with the appropriate aldehyde (II), i.e. n-butyraldehyde, in the presence of stoichiometric amounts of an inorganic base such as NaOH or Ca(OH)₂. The dimethylolbutanal formed in the first stage reacts in the second stage with the excess formaldehyde in a disproportionation reaction to give trimethylolpropane and the formate of the base used, i.e. sodium formate or calcium formate. The occurrence of these salts is a disadvantage, since they are difficult to remove from the reaction product and in addition an equivalent of formaldehyde is lost.

In the organic Cannizzaro process, a tertiary alkylamine is used instead of an inorganic base. This allows higher yields to be achieved than when using an inorganic base. Trialkylammonium formate is obtained as an undesired by-product. One equivalent of formaldehyde is accordingly likewise lost.

The disadvantages of the Cannizzaro process are avoided by the hydrogenation process. This involves reacting formaldehyde with the appropriate aldehyde (II) in the presence of catalytic amounts of an amine. This achieves the stopping of the reaction substantially at the stage of the alkylolated aldehyde. After removing the formaldehyde, the reaction mixture which, as well as the alkylolated aldehyde mentioned, still comprises small amounts of the appropriate polyhydric alcohol and of acetals of the alcohols formed is subjected to a catalytic hydrogenation to obtain the desired polyhydric alcohol.

A particular effective process for preparing polyhydric alcohols obtainable by condensation of aldehydes with formaldehyde is described in WO 98/28253. High yields combined with the occurrence of only small amounts of coupling products are facilitated by this process. The procedure is to react the higher aldehyde with the from 2- to 8-fold amount of formaldehyde in the presence of a tertiary amine and to separate the reaction mixture obtained in this manner into two solutions, one of which contains a completely methylolated alkanal mentioned and the other unconverted starting product. The latter solution is recycled into the reaction. The separation is effected by distillation or simple removal of the aqueous from the organic phase. The solution containing the product is subjected to a catalytic and/or thermal treatment in order to convert incompletely alkylolated alkanals to the desired fully methylolated compounds.

Any by-product formed is removed by distillation and the liquid phase obtained in this manner is subjected to catalytic hydrogenation which leads to the polyhydric alcohols. In the process according to the invention for preparing polyetherols, particular preference is given to using polyhydric alcohols of the formula (I) which have been obtained by the hydrogenation process, i.e. by reacting an aldehyde of the formula (II) with formaldehyde and subsequently converting the aldehyde group to a hydroxyl group by catalytic hydrogenation, more preferably those which have been obtained by the process described in WO 98/28253.

It is essential to the invention that the formaldehyde acetal content in the polyhydric alcohol used be less than 500 ppm by weight and preferably less than 400 ppm by weight.

The formaldehyde acetals (formals) are those cyclic or aliphatic compounds which comprise the structural element
—O—CH₂—O— (III)

These may be either hemiacetals or full acetals which are derived from main components and impurities, or else from by-products, intermediates or subsequent products of the reaction mixture.

These may be, for example, the following formaldehyde acetals of the formula (IV): 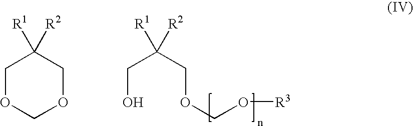
where R¹ and R² are each as defined above, and in addition
R³ is straight-chain or branched C₁-C₁₀-, preferably C₁-C₈- and more preferably C₁-C₅-alkyl, straight-chain or branched C₁-C₁₀-, preferably C₁-C₈- and more preferably C₁-C₆-hydroxyalkyl or hydrogen and
n is an integer from 1 to 4, preferably from 1 to 3 and more preferably 1 or 2.

Examples of R³ include hydrogen, methyl, ethyl, n-propyl, n-butyl, 2-methylpropyl, 2-methylbutyl, 2-ethyl-3-hydroxypropyl, 2-methyl-3-hydroxypropyl, 2,2-bis(hydroxymethyl)butyl, 2,2-bis(hydroxymethyl)propyl, 2,2-dimethyl-3-hydroxypropyl, 3-hydroxypropyl, 3-hydroxy-2-(hydroxymethyl)propyl or 3-hydroxy-2,2-bis(hydroxymethyl)propyl.

The formaldehyde acetals are preferably the following: 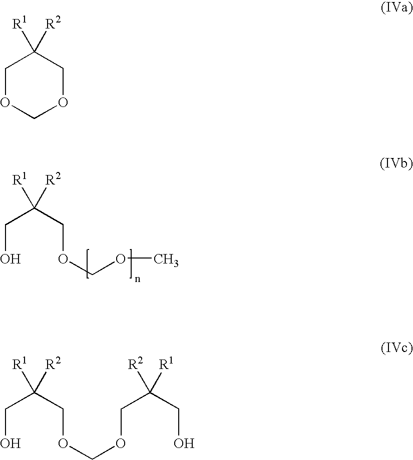
where R¹, R² and n are each as defined above.

The formaldehyde acetals are more preferably IVa, IVb (n=1), IVb (n=2) and IVc.

The methanol acetals are formed from methanol which is generally present in formaldehyde at a low level, or is formed in small amounts during the preparation by a Cannizzaro reaction of formaldehyde.

In the case of the synthesis of the trihydric alcohol trimethylolpropane (TMP) from formaldehyde and n-butyraldehyde in the presence of catalytic amounts of trialkylamine, for example, typical formaldehyde acetals are IVa, IVb (n=1), IVb (n=2) and IVc where each R¹ is ethyl and each R² is hydroxymethyl, each of which may be present in the crude product of the hydrogenation process in amounts of from 0.05 to 10% by weight.

The formaldehyde acetal content is calculated from the sum of the molar weight proportion of formaldehyde equivalents in each formaldehyde acetal multiplied by its analytically determined weight fraction in the reaction mixture.

For instance, the formaldehyde acetal content for a trimethylolpropane mixture (R¹=ethyl, R²=hydroxymethyl) which comprises the components (IVa), (IVb, where n=1 and n=2) and also (IVc), for example, is calculated as follows:

$$\begin{matrix}\begin{matrix}formaldehyde acetal \\ content [ \% by weight ]\end{matrix} = \% by weight of ( IVa ) \times \frac{30 g \text{/} mol}{146 g \text{/} mol} + \\ \% by weight of ( IVb , n = 1 ) \times \\ \frac{30 g \text{/} mol}{178 g \text{/} mol} + \% by weight of ( IVb , \\ n = 2 ) \times \frac{2 \times 30 g \text{/} mol}{208 g / mol} + \% by weight of \\ ( IVc ) \times \frac{30 g \text{/} mol}{280 g \text{/} mol}\end{matrix}$$

In order to obtain the corresponding formaldehyde acetal content in ppm by weight, this value has to be multiplied by 10 000.

The content of each component can be determined by those skilled in the art by analytical methods known per se, for example by gas chromatography or HPLC. For example, it is possible to identify each component by coupling the analytical methods mentioned with mass spectrometry.

It is irrelevant to the invention how such a low formaldehyde acetal content in the polyhydric alcohol is achieved.

U.S. Pat. No. 6,096,905 discloses a process by which a composition comprising formaldehyde acetals is treated with a strongly acidic catalyst at from 30 to 300° C. for ½ to 8 hours.

GB-A 1 290 036 describes a process by which a crude TMP solution obtained by the inorganic Cannizzaro process is treated with a cation exchanger.

A preferred process by which the formaldehyde acetal content in a polyhydric alcohol can be reduced consists in purifying the polyhydric alcohol after its preparation by distillation, then subjecting it to heat treatment and then purifying it again, preferably by distillation, as described in the German application having the reference number 100 29 055.8 and the application date Jun. 13, 2000 from BASF Aktiengesellschaft or in the international application having the title “Removal of formaldehydic acetals from polyhydric alcohols by heat treatment” of BASF Aktiengesellschaft.

When polyhydric alcohols are used in such a heat treatment step, particularly good results can be achieved when using alcohol solutions having a content of more than 60%, preferably >75%, more preferably >90%, even more preferably >95% and in particular >98%. Examples of further components of the alcohol solutions may include solvents, for example water, methanol, ethanol or n-butanol, and also by-products occurring in the preparation of the polyhydric alcohol, preferably in amounts of less than 10% by weight, more preferably in amounts of less than 5% by weight and most preferably of less than 2% by weight.

This process may be used to reduce the formaldehyde acetal content in polyhydric alcohols, preferably those alcohols of the formula (I) and in particular trimethylolpropane of any origin. Charges may be used which result from the organic or the inorganic Cannizzaro process. The best results were obtained when alcohols which stem from the hydrogenation process were used in the process serving to reduce the formaldehyde acetal. In any case, it is important that the alcohol has been previously purified and has a purity in the abovementioned range.

When the process is to be used to remove formaldehyde acetals from crude solutions of polyhydric alcohols, in particular of trimethylolpropane, having product contents of from 60 to 95% by weight, preference is given to subjecting the crude product obtained after the hydrogenation process (hydrogenation effluent) before the heat treatment step to dewatering in which the water and other low boilers such as methanol and trialkylamine or trialkylammonium formate are removed by distillation.

In order to achieve the desired reduction in the formaldehyde acetal content in this process, certain reaction conditions have to be maintained which may vary depending, for instance, on the type of polyhydric alcohol used, the purity of the products used, the apparatus used and any further components or additives present. These reaction conditions may be obtained by those skilled in the art by experiments.

In general, the heat treatment step is carried out at temperatures of from 100 to 300° C., preferably from 160 to 240° C., at residence times of from 5 min to 24 h, preferably from 15 min to 4 h and at pressures from 100 mbar to 200 bar, preferably from 1 to 10 bar.

When the polyhydric alcohol to be purified is trimethylolporpane, the heat treatment step is carried out at temperatures from 100 to 300° C., preferably from 160 to 240° C., residence times of 10 min to 24 h, preferably from 1 h to 5 h, more preferably from 30 min to 6 h and most preferably from 45 min to 4 h, and at the abovementioned pressures.

To carry out the heat treatment step, the customary apparatus known to those skilled in the art may be used continuously or batchwise. In batchwise operation, preference is given to carrying out the heat treatment step in a stirred vessel, and in the batchwise procedure in a tubular reactor employing either the liquid phase or trickle method.

The most preferred embodiment of the heat treatment step is the continuous operation in a tubular reactor in the liquid phase method.

In all these operation variants, the reaction vessel may be provided with the customary dense packings known to those skilled in the art, for example Raschig or Pall rings, or with structured packings, for example sheet metal packings, in order to achieve better mixing of the components. Supports and/or catalysts may also be present in the customary forms, for example extrudates or tablets, in order to accelerate the reactions proceeding in the heat treatment step. Examples of suitable supports/catalysts include TiO₂, Al₂O₃, SiO₂, supported phosphoric acid (H₃PO₄) and zeolites.

In one variant of the heat treatment step, a suitable additive is added to the reaction solution during the heat treatment step in order to accelerate and ease the reactions leading to the reduction in the amounts of formaldehyde acetals. Examples thereof include not too strong and/or reducing acids or their anhydrides or ion exchangers, as described in U.S. Pat. No. 6,096,905 or GB 1 290 036. Examples of suitable acids include phosphoric acid, phosphorous acid, hypophosphorous acid, boric acid, carbonic acid and sulfurous acid. Gases, for example CO₂ and SO₂, which react acidically in aqueous solution, are also suitable.

The acids to be used as additives are used in amounts of from 10 ppm to 1% by weight, preferably from 100 to 2000 ppm. Since the additive possibly added has to be removed from the formaldehyde acetal-reduced polyhydric alcohol after the heat treatment step, preference is given to this additive being gaseous and accordingly being easy to remove from the reaction mixture by outgassing.

It may further be advantageous to carry out the heat treatment step for decomposing the formaldehyde acetals under an inert gas, for example nitrogen, argon or helium, preferably under nitrogen.

Without wishing to be bound to a theory, it is suspected that formaldehyde acetals are converted by the heat treatment step in the alcohol prepurified by distillation into higher-boiling, involatile and low-boiling components and can thus be distillatively removed more easily.

The polyhydric alcohol having a reduced formaldehyde acetal content can be easily removed from the high-boiling involatile components formed by distillation. The heat treatment step is therefore generally followed by a distillation. Since the involatile components formed from the formaldehyde acetals in the heat treatment step generally differ markedly from the polyhydric alcohols with regard to their boiling behavior, these may be removed by simple distillative measures or methods having only a small separating effect. Separating units having only one distillation stage, for example falling-film evaporators or thin-film evaporators, often suffice. Particularly when the distillation also serves for further purification of the product alcohol, more complicated separating processes or separating apparatus may optionally be used, generally columns having more than one separating stage, for example randomly packed columns, bubble cap tray columns or columns having structured packing.

The distillation is carried out using the customary conditions with regard to pressure and temperature known to those skilled in the art, although it will be appreciated that these also depend on the product alcohol used. According to a further embodiment, the heat treatment step may also be combined with the distillation. In this embodiment, the heat treatment takes place in the column bottom of the distillation apparatus in which the polyhydric product alcohol is removed from involatile components formed in the heat treatment and also any other impurities. When the heat treatment step and distillation are combined in one stage, it is important that the above-specified reaction conditions with regard to pressure, temperature and in particular residence time are maintained in order to achieve sufficient decomposition of the formaldehyde acetals. When the heat treatment and distillation steps are combined in a single process step, preference is given to adding acid.

The polyhydric alcohol obtainable by this process generally has a formaldehyde acetal content as defined above of less than 500 ppm by weight, preferably less than 400 ppm by weight.

It is unimportant by which process the polyhydric alcohol has been obtained, for example by the Cannizzaro or by the hydrogenation process.

To prepare the alkoxylated alcohols (polyetherols), a polyhydric alcohol having a formaldehyde acetal content of less than 500 ppm is reacted with at least one alkylene oxide.

Examples of useful alkylene oxides include ethylene oxide, propylene oxide, iso-butylene oxide, vinyloxirane and/or styrene oxide, and preference is given to ethylene oxide, propylene oxide and/or iso-butylene oxide, particular preference to ethylene oxide and/or propylene oxide.

Preferred examples of such alkoxylated alcohols are the alkoxylation products (Va), (Vb) or (Vc) of alcohols of the formula (I) 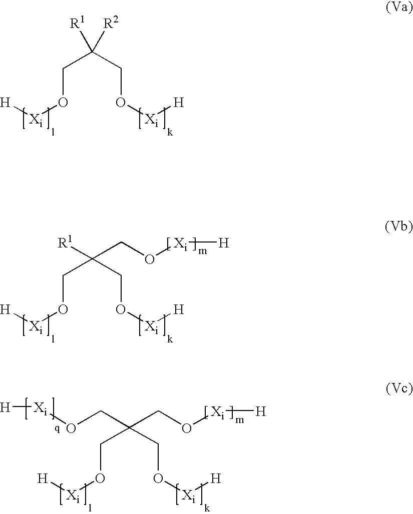
where
R¹ and R² are each as defined above,
k, l, m and q are each integers from 1 to 50, preferably from 3 to 20 and
each Xᵢ for i=1 to k, 1 to 1, 1 to m and 1 to q is in each case independently selected from the group of —CH₂—CH₂—O—, —CH₂—CH(CH₃)—O—, —CH(CH₃)—CH₂—O—, —CH₂—C(CH₃)₂—O—, —C(CH₃)₂—CH₂—O—, —CH₂—CHVin-O—,
   —CHVin-CH₂—O—, —CH₂—CHPh-O— and —CHPh-CH₂—O—, preferably from the group of —CH₂—CH₂—O—, —CH₂—CH(CH₃)—O— and —CH(CH₃)—CH₂—O—,
      where Ph is phenyl and Vin is vinyl.

The alkoxylated alcohol is preferably neopentyl glycol, trimethylolpropane, trimethylolethane or pentaerythritol, each of which has been ethoxylated, propoxylated or partly ethoxylated and partly propoxylated from 1 to 20 times, more preferably from 3 to 19 times.

Among these, particular preference is given to those polyhydric alcohols of the formula (Vb).

The reaction of the alcohols with an alkylene oxide is known per se to those skilled in the art. Possible embodiments can be found in Houben-Weyl, Methoden der Organischen Chemie, 4th edition, 1979, Thieme Verlag Stuttgart, Ed. Heinz Kropf, Volume 6/1a, Part 1, pages 373 to 385.

Preference is given to carrying out the reaction as follows:

The polyhydric alcohol is initially charged, optionally in a suitable solvent, at temperatures of from 0 to 200° C., preferably from 100 to 180° C., preferably under protective gas, for example nitrogen. To this end, the alkylene oxide, optionally at a temperature of from −30 to 50° C. and dissolved in one of the abovementioned solvents, is added continuously or in portions with good mixing in such a manner that the temperature of the reaction mixture is maintained between 100 and 180° C., preferably between 100 and 150° C. The reaction may take place under a pressure of up to 60 bar, preferably of up to 30 bar and more preferably of up to 10 bar.

Such an amount of alkylene oxide is used that up to (1.1×(k+l+m+q)) mol of alkylene oxide, preferably up to (1.05×(k+l+m+q)) mol of alkylene oxide and more preferably (k+l+m+q) mol of alkylene oxide, are used per mole of polyhydric alcohol, where k, l, m and q are each as defined above.

Optionally, up to 50 mol % based on the polyhydric alcohol, more preferably up to 25 mol % and most preferably up to 10 mol %, of a catalyst may be added for acceleration, for example monoethanolamine, diethanolamine, triethanolamine, dimethylaminoethanolamine, ethylene glycol or diethylene glycol, or else alkali metal hydroxides or alkoxides, or hydrotalcite, preferably alkali metal hydroxides in water. When DMC catalysts are used, they are preferably added in an amount of from 10 to 1000 ppm, preferably from 10 to 500 ppm and in particular from 10 to 250 ppm.

After the complete metering in of the alkylene oxide, the reaction is generally allowed to continue for from 10 to 500 min, preferably from 10 to 120 min, at temperatures of from 30 to 220° C., preferably from 80 to 200° C. and more preferably from 100 to 180° C., and the temperature may remain the same or be raised in stages or continuously.

The conversion of alkylene oxide is preferably at least 90%, more preferably at least 95% and most preferably at least 98%. Any residues of alkylene oxide may be stripped out of the reaction mixture by passing through a gas, for example nitrogen, helium, argon or steam.

The reaction may be carried out batchwise, semibatchwise or continuously in a stirred reactor or else continuously in a tubular reactor having static mixers.

Preference is given to carrying out the reaction completely in the liquid phase.

The reaction product formed may be further processed in crude or worked-up form.

When further processing in pure form is desired, the product may be purified, for example, by crystallization and solid/liquid separation.

Customarily, the base is neutralized and salted out by adding an acid. The acids used are organic acids such as formic acid or acetic acid or dilute inorganic acids such as phosphoric acid, hydrochloric acid or sulfuric acid.

The yields are generally over 75%, usually over 80% and frequently over 90%.

The process by which the preparation of polyetherols from alkylene oxides and a polyhydric alcohol is carried out is not restricted. It is essential to the invention that the polyhydric alcohol used has a formaldehyde content as defined above of less than 500 ppm by weight, preferably 400 ppm.

The polyether alcohols prepared by the process according to the invention may be used, for example, as lubricants or further processed. For example, the polyether alcohols may be further processed to acrylates or, by reaction with isocyanates, to polyurethanes.

Unless otherwise stated, ppm and percentage data used in this document refer to percent by weight and ppm by weight.

APHA color numbers were determined to DIN-ISO 6271.

The invention is illustrated by the examples hereinbelow.

Preparation Method of the Polyetherols

The gas chromatography determination of the formaldehyde acetal contents quoted in this application was carried out using the column DB5 of length 30 m, diameter 0.32 mm and coating thickness 1 μm. Detection was effected using a flame ionization detector. The formaldehyde acetal content determined is referred to hereinbelow as the formaldehyde number.

In a stirred tank, 1090 liters of molten TMP having different formaldehyde numbers were initially charged at 100° C. and 8.2 liters of a 45% potassium hydroxide solution were added under nitrogen. By heating to 120° C. at a pressure of 20 mbar, the water was distilled off, then nitrogen was injected and at an oxygen content of <0.3%, a total of 7520 liters of ethylene oxide were gradually injected into the solution at a temperature of 160° C. After one hour at 160° C., the mixture was cooled to 90° C. and aerated with nitrogen. 203 liters of demineralized water were then added and the mixture stirred at 90° C. for 1 hour. To demineralize the polyetherol, 9.9 liters of 35% phosphoric acid were then added, and the mixture was stirred at 90° C. for 30 minutes and filtered. To completely remove water, the polyetherol was distillatively dewatered at 120° C. and 20 mbar for one hour and cooled.

In the table, experiments using different TMP qualities and the resulting color numbers are summarized.

| | Formaldehyde number | Color number of the |
|---|---|---|
| | of the TMP used | polyetherol |
| Experiment No. | [ppm by weight] | [APHA] |
| I | 280 | 10 |
| II | 460 | 17 |
| III | 620 | 26 |
| IV | 860 | 61 |
| V | 1400 | 289 |

1. A process for preparing a polyetherol of a polyhydric alcohol by reacting an alkylene oxide with the appropriate polyhydric alcohol in the presence of a base and in the presence or absence of a solvent, wherein the polyhydric alcohol has been obtained by reacting an aldehyde of the formula (II) 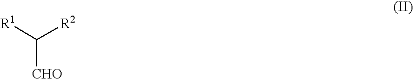
where R¹ and R² are each independently hydrogen, C₁-C₁₀-alkyl, C₁-C₁₀-hydroxyalkyl, carboxyl or C₁-C₄-alkoxycarbonyl with formaldehyde and then converting the aldehyde group to a hydroxyl group by catalytic hydrogenation and has a formaldehyde acetal content of less than 500 ppm.

2. The process as claimed in claim 1, wherein the polyhydric alcohol is an alcohol of the formula (I) 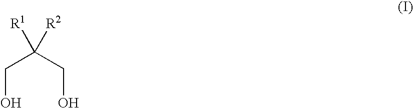
where R¹ and R² are each independently hydrogen, C₁-C₁₀-alkyl, C₁-C₁₀-hydroxyalkyl, carboxyl or C₁-C₄-alkoxycarbonyl.

3. The process as claimed in claim 1, wherein the polyhydric alcohol is trimethylolbutane, trimethylolpropane, trimethylolethane, neopentyl glycol, pentaerythritol, 2-ethyl-1,3-propanediol, 2-methyl-1,3-propanediol, 1,3-propanediol, dimethylolpropionic acid, methyl dimethylolpropionate, ethyl dimethylolpropionate, dimethylolbutyric acid, methyl dimethylolbutyrate or ethyl dimethylolbutyrate.

4. The process as claimed in claim 1, wherein the polyhydric alcohol, is purified by distillation, then subjected to heat treatment and then purified again.

5. A process as claimed in claim 4, wherein the heat treatment is carried out at from 100 to 300° C.

6. The process as claimed in claim 1, wherein the molar ratio of the number of hydroxyl groups in the polyhydric alcohol to the alkylene oxide is from 0.25 to 50.

7. The process as claimed in claim 1, wherein the base used in the etherification is monoethanolamine, diethanolamine, triethanolamine, dimethylaminoethanolamine, or an alkali metal hydroxide or alkoxide or hydrotalcite, an alkali metal hydroxide in water or a mixture thereof.

8. (canceled)

9. A method of etherifying an alkylene oxide, said method comprising etherifying said alkylene oxide with a polyhydric alcohol of formula (I) 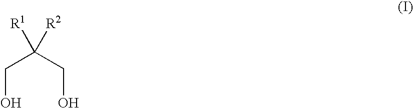
where R¹ and R² are each independently hydrogen, C₁-C₁₀-alkyl, C₁-C₁₀-hydroxyalkyl, carboxyl or C₁-C₄-alkoxycarbonyl;
wherein said polyhydric alcohol has a formaldehyde acetal content of less than 500 ppm.
